(19) United States
(12) Patent Application Publication
(10) Pub. No.: US 2018/0125816 A1
(43) Pub. Date: May 10, 2018

(54) SAROGLITAZAR MAGNESIUM FOR THE TREATMENT OF CHYLOMICRONEMIA SYNDROME

(71) Applicant: CADILA HEALTHCARE LIMITED, Ahmedabad, Gujarat (IN)

(72) Inventors: Mukul R. JAIN, Ahmedabad - Gujarat (IN); Suresh GIRI, Ahmedabad - Gujarat (IN)

(73) Assignee: CADILA HEALTHCARE LIMITED, Ahmedabad, Gujarat (IN)

(21) Appl. No.: 15/572,538

(22) PCT Filed: May 10, 2016
(86) PCT No.: PCT/IN2016/000121

§ 371 Date: Nov. 8, 2017

(30) Foreign Application Priority Data
May 11, 2015 (IN) 1850/MUM/2015

(51) Int. Cl. A61K 31/40; A61P 3/06

(52) U.S. Cl. CPC A61K 31/40; A61P 3/06

(57) ABSTRACT

The present invention relates to the use of Saroglitazar Magnesium of formula (1), for the prevention, delay of progression, or treatment of a disease or condition which is selected from chylomicronemia syndrome, familial chylomicronemia syndrome and Type V hyperlipoproteinemia. The present invention further relates to the use of a pharmaceutical composition comprising Saroglitazar Magnesium for the prevention, delay of progression, or treatment of a disease or condition which is selected from chylomicronemia syndrome, familial chylomicronemia syndrome and Type V hyperlipoproteinemia.

### FIELD OF INVENTION

The present invention relates to the use of Saroglitazar Magnesium of formula (1), for the treatment of, or the prevention, delay of progression, or treatment of a disease or condition which is selected from chylomicronemia syndrome, familial chylomicronemia syndrome and Type V hyperlipoproteinemia. The present invention further relates to the use of a pharmaceutical composition comprising Saroglitazar Magnesium for the prevention, delay of progression, or treatment of a disease or condition which is selected from chylomicronemia syndrome, familial chylomicronemia syndrome and Type V hyperlipoproteinemia.

### BACKGROUND OF INVENTION

Hyperlipidemia, or the presence of elevated levels of lipids in the bloodstream, can take the form of hypercholesterolemia (elevated cholesterol), hypertriglyceridemia (elevated triglyceride) or a combination of the two. Hypercholesterolemia, which can further be subdivided, is typically associated with increased risk of atherosclerosis cardiovascular disease. Hypertriglyceridemia occurs when the body's production or intake of triglyceride exceeds the body's ability to metabolize or remove the triglyceride from the blood stream. The most severe form of hypertriglyceridemia is chylomicronemia (also called hyperchylomicronemia), and is associated with an increased risk of pancreatitis. Chylomicrons are lipoprotein particles that carry absorbed dietary fat from the gut to other body tissues via the bloodstream, and are typically present only during meal times. Chylomicronemia is defined as having the presence of chylomicrons in the blood stream during times of fasting, and is typically associated with total plasma triglyceride levels above 1000 mg/dl.

The chylomicronemia syndrome refers to a set of clinical complications associated with high chylomicron levels. Typically, patients with the chylomicronemia syndrome have markedly elevated fasting triglyceride levels (1000-2000 mg/dL), with profound excursions (up to 5000 mg/dL and higher) following oral fat intake. The massively elevated plasma triglyceride levels are associated with a number of clinical findings and complications including recurrent episodes of pancreatitis, deposition of triglycerides in the skin in the form of eruptive xanthomas, hepatosplenomegaly, a milky pink appearance of the blood vessels in the back of the eye (lipemia retinalis), and mild neuro-cognitive deficits.

The chylomicronemia syndrome can be further sub divided into two groups based on ultracentrifugation of lipoprotein species (see “A system for phenotyping hyper lipoproteinemia”, Fredrickson D. S., Lees R. S. Circulation, 1965 March; 31, pp. 321-327). Fredrickson classification Type I, also known as the familial chylomicronemia syndrome (FCS), patients have accumulation of only chylomicrons in the bloodstream whereas Fredrickson classification Type V, also known as Type V hyperlipoproteinemia, patients have accumulation of both chylomicrons and very low density lipoproteins (VLDL) in the bloodstream.

The familial chylomicronemia syndrome (FCS or Type I hyperlipoproteinemia) is caused by a homozygous or compound heterozygous defect in the clearance of chylomicrons from the bloodstream. The most common cause of FCS is a defect in lipoprotein lipase (LPL), the protein that hydrolyzes triglycerides carried on chylomicrons. Other causes of FCS include defects in apolipoprotein CH (apoCll, a co activator of LPL) or glycosylphosphatidylinositol-anchored high-density lipoprotein-binding protein 1 (GPlHBP1, an anchoring protein of LPL).

Type 1 patients are usually identified by early onset as youth of hypertriglyceridemia and pancreatitis. Thus, patients With FCS typically present in childhood with massively elevated triglyceride levels (>2,000 mg/dL), and recurrent bouts of abdominal pain due to pancreatitis. Into adult hood, the triglyceride levels remain elevated, and patients typically experience multiple episodes of abdominal pain and pancreatitis, which can result in hospitalization and death. Patients also experience other manifestations including eruptive xanthomas, lipemia retinalis, hepatosplenomegaly, and mild neuro-cognitive deficits. The main therapeutic goal in FCS treatment is to prevent or treat pancreatitis via the reduction of triglycerides.

Unfortunately, standard lipid-lowering therapies, such as fibrates, omega-3 fatty acids, statins, and nicotinic acid derivatives (niacin), are not effective in lowering triglycerides in patients with FCS. Therefore, the standard of care therapy for FCS patients is a very low fat diet (10% by calories), something which is very difficult to stay compliant with throughout a lifetime [The Familial Chylomicronemia Syndrome. Santamarina-Fojo S. Lipid Disorders 1998. 27(3): 551-567].

At present there is thus no effective pharmaco therapy for treating PCS and there is thus a need for new methods of treating familial chylomicronemia syndrome (PCS), also known as Type I hyperlipoproteinemia.

Type V hyperlipoproteinemia patients represent a second group at risk for the chylomicronemia syndrome and are usually diagnosed by severe hypertriglyceridemia as adults. This is a heterogenous group at the extreme end of a spectrum of multifactorial hypertriglyceridemia. Patients with type V hyperlipoproteinemia generally have both an underlying genetic cause and one or more acquired causes of hypertriglyceridemia. The underlying genetic causes include well characterized dyslipidemia such as familial combined hyperlipidemia (Type HA), dysbetalipoproteinemia (Type III) and familial hypertriglyceridemia (Type VI), and a group of less well characterized dyslipidemias (e.g. heterozygous LPL deficiency, defects in apoA & apoC genes, defects in fatty acid binding and transport proteins). Acquired causes of hypertriglyceridemia include comorbid diseases (eg type 2 diabetes, obesity, insulin resistance, lipodystrophy, hypothyroidism), medications (e.g. beta blockers, thiazide diuretics, estrogen, glucocorticoids, transplant medications), and other factors (e.g. pregnancy, alcohol intake).

The primary goal of therapy in Type V patients is to reduce the triglyceride levels, and therefore reduce the risk of pancreatitis. Most patients can be successfully treated by addressing the underlying acquired cause(s) of the elevated triglycerides, such as reducing the amount of dietary fat intake, treating uncontrolled co-morbid diseases such as T2DM (Type 2 diabetes mellitus), discontinuing offending medications, and initiating lipid lowering medications such as fibrates, omega-3 fatty acids, or nicotinic acid derivatives (niacin) [Chylomicronemia Syndrome. Chait A., BrunZell J. Adv Intern Med 1992. 37:249-73.]. Despite optimal therapy, some Type V patients continue to have elevated triglyceride levels. There is thus a need for new methods of treating Type V hyperlipoproteinemia, especially for those patients whose triglyceride levels remain elevated even after treatment with conventional therapies.

WO 2011123401 discloses use of certain DGAT1 inhibitors, or their pharmaceutically acceptable salts or esters, for the prevention, delay of progression or treatment of a disease or condition which is selected from chylomicronemia syndrome, familial chylomicronemia syndrome and Type V hyperlipoproteinemia. WO 2013169648 describes a pharmaceutical combination, such as a combined preparation of two pharmaceutical composition, comprising at least one DGAT1 inhibitor, or a pharmaceutically acceptable salt or ester thereof and at least one kind of triglyceride lowering drug selected from the group consisting of (a) at least one PPAR alpha agonist or a pharmaceutically acceptable salt thereof or ester thereof, and (b) at least one compound selected from the group consisting of (i) natural or synthetic omega-3 fatty acids or pharmaceutical acceptable esters, derivatives, conjugates, precursors or salts thereof or mixtures thereof, or (ii) omega-3 oils, and optionally at least one pharmaceutically acceptable carrier for simultaneous, separate or sequential use, in particular for the treatment of hypertriglyceridemia, in particular chylomicronemia (also called hyperchylomicronemia)

WO 2013163508 describes tetrahydropyran DGAT1 inhibitors for use in the prevention, delay of progression or treatment of a disease or condition which is selected from chylomicronemia syndrome, familial chylomicronemia syndrome, and Type V hyperlipoproteinemia.

WO 2011079257 describes a fully human antibody or antigen-binding fragment of a human antibody that specifically binds and inhibits human angiopoietin-like protein 4 (hANGPTL4) which are useful in treating diseases or disorders associated with ANGPTL4, such as hyperlipidemia, hyperlipoproteinemia and dyslipidemia, including hypertriglyceridemia, hypercholesterolemia, chylomicronemia.

WO 2012162129 describes some other novel DGAT1 inhibitors for use in the prevention, delay of progression or treatment of a disease or condition which is selected from chylomicronemia syndrome, familial chylomicronemia syndrome, and Type V hyperlipoproteinemia

WO 2013106358 describes novel role for microRNA (miR) regulation of lipid metabolism via the MTP pathway, leading to reductions in apoB secretion and blood lipid levels. It describes inhibition of MTP expression and activity by miR regulation as a new therapeutic target for treatment of cardiovascular disease and conditions or diseases associated with cardiovascular disease such as hyperlipidemia, atherosclerosis, and metabolic syndrome.

EP 1725234 describes methods and compositions for treating hyperlipidemia and/or hypercholesterolemia comprising administering to the subject an effective amount of an MTP inhibitor to inhibit hyperlipidemia and/or hypercholesterolemia in said subject.

WO 2014205449 describes compounds, compositions and associated methods for reducing expression of ApoCIII mRNA and protein in a subject having, or at risk of having, diabetes.

WO 2014019919 discloses pharmaceutical compositions comprising 15-OHEPA and, optionally, one or more cardiovascular agents, as well as therapeutic methods for treating various diseases including homozygous familial chylomicronemia, hypercholesterolemia, hyperlipidemia, hyperlipidemia in HIV positive subjects.

The present invention describes the use of a PPAR modulator of formula (1) for the treatment of familial chylomicronemia, hypercholesterolemia, hyperlipidemia, hyperlipidemia in patients in need thereof. It has surprisingly been found that Saroglitazar Magnesium of formula (1) is useful for the treatment of the above conditions alone, unlike those reported in the prior art where the PPAR agonists had to be used in combination with a second therapeutic agent.

### SUMMARY OF THE INVENTION

The compound of formula (1) is approved for the treatment of treating diabetic dyslipidemia or hypertriglyceridemia in type 2 diabetes, not controlled by statins alone.

The present inventors have surprisingly found that Saroglitazar Magnesium of formula (1), reduces postprandial triglyceride levels in animal models. Obese Zucker (fatty) rat is one of the oldest models of NIDDM (Non-insulin dependent diabetes mellitus), resembling human type II diabetes, i.e. diabetes associated with obesity. It is characterized by hyperphagia and associated with mild hyperglycaemia, insulin resistance, mild glucose intolerance, hyperlipidaemia specially hypertriglyceridemia. When the non-ionic detergent Triton WR-1339 is injected into Zucker fa/fa rats, there is a progressive accumulation of triacylglycerols in the plasma/serum (Friedman & Byers, J Exp Med. 1953 January; 97(1):117-30; Otway & Robinson, J Physiol. 1967 May; 190(2):321-32.). This effect has been interpreted as being due to the inability of lipoprotein lipase present in the extrahepatic tissues to hydrolyse the plasma triacyl glycerols of Triton-treated animals (Scanu & Oriente, JEM vol. 113 no. 4 735-757 1961; Otway & Robinson, 1967). So the nonionic detergent, Triton WR-1339 (Triton), has commonly been used to inhibit LPL for this purpose to produce the condition of severe hypertriglyceridemia similar to familial chylomicronemia syndrome and Type V hyperlipoproteinemia in humans. The Compound (1) was also tested for hyper-chylomicronemia and chylomicron dynamics and tissue uptake, distribution and recycling of lipids in vivo by using ¹³C-palmitate in Zucker fa/fa rats. (ref.: PNAS, vol. 112, no. 4, 1143-1148; Journal of lipid Research, vol. 38, 1997, 1888-1895). The results demonstrate metabolic defects, which although exaggerated, closely parallel the defects in postprandial handling of plasma lipids in patients with chylomicronemia syndrome (including patients with familial chylomicronemia syndrome and patients With Type V hyperlipoproteinemia).

The present invention thus provides Saroglitazar Magnesium for use in the prevention, delay of progression, or treatment of a disease or condition which is selected from chylomicronemia syndrome, familial chylomicronemia syndrome, and Type V hyperlipoproteinemia.

There is also provided Saroglitazar Magnesium for use in the reduction of postprandial triglyceride levels in patients suffering from a disease or condition which is selected from chylomicronemia syndrome, familial chylomicronemia syndrome, and Type V hyperlipoproteinemia.

There is also provided Saroglitazar Magnesium for use in the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits.

In another embodiment, there is provided Saroglitazar Magnesium for use in the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, in patients suffering from a disease or condition which is selected from chylomicronemia syndrome, familial chylomicronemia syndrome, and Type V hyperlipoproteinemia.

In another aspect of the invention, there is provided a pharmaceutical composition, comprising Saroglitazar Magnesium along with suitable excipients for use in the prevention, delay of progression or treatment of a disease or condition which is selected from chylomicronemia syndrome, familial chylomicronemia syndrome, and Type V hyperlipoproteinemia.

There is also provided a pharmaceutical composition, comprising compound (1), for use as defined above.

In another aspect, there is also provided a method for the prevention, delay of progression, or treatment of a disease or condition which is selected from chylomicronemia syndrome, familial chylomicronemia syndrome, and Type V hyperlipoproteinemia, comprising administration of a therapeutically effective amount, or a prophylactically effective amount, of compound (1) to a subject, e. g. a human subject, in need of such treatment.

There is also provided a method for the reduction of postprandial triglyceride levels in patients suffering from a disease or condition which is selected from chylomicronemia syndrome, familial chylomicronemia syndrome, and Type V hyperlipoproteinemia, comprising administration of a therapeutically effective amount of compound (1) to a subject, e.g. a human, in need of such treatment.

There is also provided a method for the prevention, delay of progression or treatment of pancreatitis in patients suffering from a disease or condition which is selected from chylomicronemia syndrome, familial chylomicronemia syndrome, and Type V hyperlipoproteinemia, comprising administration of a therapeutically effective amount of the compound (1), to a subject, e.g. a human, in need of such treatment.

There is also provided a method for the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, comprising administration of a therapeutically effective amount of compound (1), to a subject, e.g. a human, in need of such treatment.

In another embodiment, there is provided a method for the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, comprising administration of a therapeutically effective amount of compound (I) to a human subject suffering from a disease or condition Which is selected from chylomicronemia syndrome, familial chylomicronemia syndrome, and Type V hyperlipoproteinemia.

### DETAILED DESCRIPTION

The present invention thus provides the use of Saroglitazar Magnesium of formula (1), for the manufacture of a medicament for the prevention, delay of progression, or treatment of a disease or condition which is selected from chylomicronemia syndrome, familial chylomicronemia syndrome, and Type V hyperlipoproteinemia.

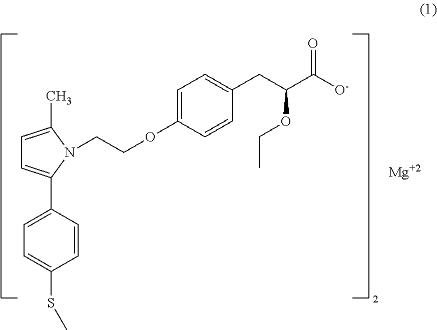

There is also provided the use of the Compound (1) for the manufacture of a medicament for one or more of the following purposes: (a) the reduction of postprandial triglyceride levels in patients suffering from a disease or condition which is selected from chylomicronemia syndrome, familial chylomicronemia syndrome, and Type V hyperlipoproteinemia; (b) the prevention, delay of progression or treatment of pancreatitis in patients suffering from a disease or condition which is selected from chylomicronemia syndrome, familial chylomicronemia syndrome, and Type V hyperlipoproteinemia: (c) the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits; (d) the prevention, delay of progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, in patients suffering from a disease or condition Which is selected from chylomicronemia syndrome, familial chylomicronemia syndrome, and Type V hyperlipoproteinemia.

The present invention also provides the suitable pharmaceutical composition of compounds of formula (1) or their derivative. The pharmaceutical composition of the present invention essentially comprises of:
the pharmaceutically active substance;
a suitable buffering agent;
a suitable stabilizer;
optionally with one or more pharmaceutically acceptable excipients.

The suitable stabilizers used in pharmaceutical composition are selected from Polacrilin potassium, Potassium chloride, Sodium stearyl fumarate and preferably selected from Sodium stearyl fumarate. The suitable buffering agent are selected from sodium acetate, ammonia solution, ammonium carbonate, sodium borate, adipic Acid, glycine, monosodium glutamate and preferably selected from ammonia solution.

The pharmaceutically acceptable excipients are selected at least one from carriers, binders, antioxidant agents, disintegrating agents, wetting agents, lubricating agents, chelating agents, surface active agents, and the like.

Diluents include, but are not limited to lactose monohydrate, lactose, polymethacrylates selected from Eudragit, potassium chloride, sulfobutylether b-cyclodextrin, sodium chloride, spray dried lactose, and preferably sulfobutyl ether b-cyclodextrin. Carriers include, but are not limited to lactose, white sugar, sodium chloride, glucose, urea, starch, calcium carbonate and kaolin, crystalline cellulose, and silicic acid. Binders include, but are not limited to carbomers selected from carbopol, gellan, gum Arabic, hydrogenated vegetable oil, polymethacrylates selected from Eudragit, xanthan, lactose and Zein. Antioxidant agents include, but are not limited to, Hypophosphorous acid, Sodium formaldehyde, sodium formaldehylde sulfoxylate, sulfur dioxide, tartaric acid, thymol and methionine. Disintegrating agents include, but are not limited to, bicarbonate salt, chitin, gellan gum, polacrillin potassium and Docusate Sodium. Wetting agents include, but are not limited to, Glycerin, lactose, Docusate Sodium and Glycine, Lubricating agents used include, but are not limited to, Glycerin behenate, hydrogenated vegetable oil, sodium stearyl fumarate and Myristic Acid, Chelating agents include, but are not limited to, Maltol and Pentetic Acid. Surface active agents include but are not limited to, Nonionic surfactant selected from alkyl polyglucosides, cocamide DEA, cocamide MBA, cocamide TEA, decyl maltoside and octyl glucoside; Anionic surfactant selected from arachidic acid and arachidonic acid; Cationic surfactant selected from cetyl trimethylammonium bromide and cetylpyridinium chloride.

The following studies were conducted in suitable animal models as described hereinafter.

### Experiment 1

### Effect of Compound (1) on Plasma Triglycerides in a Model of Severe Hypertriglyceridemia Induced by Triton WR-1399 (by Inhibition of LPL) in Zucker fa/fa Rats

The obese Zucker (fatty) rat is one of the oldest models of NIDDM, resembling human type II diabetes, i.e. diabetes associated with obesity. It is characterized by hyperphagia and associated with mild hyperglycaemia, insulin resistance, mild glucose intolerance, hyperlipidaemia specially hypertriglyceridemia. When the non-ionic detergent Triton WR-1339 is injected into Zucker fa/fa rats, there is a progressive accumulation of triacylglycerols in the plasma/serum (Friedman & Byers, 1953; Otway & Robinson, 1967). This effect has been interpreted as being due to the inability of lipoprotein lipase present in the extrahepatic tissues to hydrolyse the plasma triacylglycerols of Triton-treated animals (Scanu & Oriente, 1961; Scanu et al., 1961; Otway & Robinson, 1967). So the nonionic detergent, Triton WR-1339 (Triton), has commonly been used to inhibit LPL for this purpose to produce the condition of severe hypertriglyceridemia similar to familial chylomicronemia syndrome and Type V hyperlipoproteinemia.

In this study 20 male and 14 female Zucker fa/fa rats of 6-10 weeks of age were selected for the and on day-0, body weight of animals recorded and blood samples (approximately 80 μl to 90 μl) was collected via retro orbital route using isoflurane as inhalation anaesthesia, serum separated and serum triglyceride estimated and 24 animals were selected and grouped on the basis of serum triglyceride level as primary parameter as below

TABLE NO. 1

| Sr. | | Dosage | | Number of |
|---|---|---|---|---|
| no | Treatment Group | Level | Sex | Animals |
| 1 | Vehicle Control [5% PEG400 + | 0 mg/kg | 3F + 3M | 6 |
| | 5% tween 80 + 90% | | | |
| | Na-MC(0.5%)], p.o. | | | |
| 2 | Compound (1)-1 mg/kg, p.o. | 1 mg/kg | 3F + 3M | 6 |
| 3 | Compound (1)-10 mg/kg, p.o. | 10 mg/kg | 2F + 4M | 6 |
| 4 | Fenofibrate 65 mg/kg, p.o. | 65 mg/kg | 3F + 3M | 6 |

From hyphen or day-1 to Day-14, compounds were formulated in 5% PEG+5% Tween 80+90% (0.5%) Na-CMC in such, a way that the final volume of administration was 2 ml/kg of body wt, and animals were dosed orally once daily as per grouping details.

On day-14 blood samples were collected after 1 hour of dosing, serum separated and serum triglycerides (TG) was estimated. Animals were being kept on 24 hours fasting after completion of day-14 activities. On day-15, after 24 hours of fasting, body wt. of all animals was recorded. Then formulations of treatment items were prepared in vehicle and Triton WR-1339 prepared in 0.9% saline for intraperitoneal injection at dose of 250 mg/kg per 5 ml. Basal blood samples collected and animals were administered orally with respective treatment and immediately injected intraperitoneally with Triton WR-1339. Blood samples (approx, 1 ml per sample) were also collected at 6 hr, 24 hours and 48 hours after triton administration and serum triglycerides levels were estimated using Cobas C 311 clinical chemistry autoanalyser from Roche Diagnostics using commercial diagnostic kits.

Calculation:

% change at 6-hr, 24-hr and 48-hr for TG vs Vehicle Control was calculated using MS using MS Excel. The area under curve for TG, AUC-TG (0-48), was calculated using Graph Pad® software.

Results

The serum triglycerides levels in different treatment groups (Table no. 2) and % change Vs Vehicle Control (Table no. 3) after Triton WR-1339 administration is depicted in below tables—

TABLE NO. 2

| | Serum Triglycerides (mg/dl) at various time point after | | | | |
|---|---|---|---|---|---|
| | Triton WR-1339 administration | | | | AUC-TG (0-48 hr.) |
| Treatment Groups | 0 hr | 6 hr | 24 hr | 48 hr | mg/dl · hr |
| Vehicle Control | 519.2 ± 72.2 | 777.2 ± 53.6 | 3250.6 ± 485.6 | 2172.7 ± 555.9 | 105218.0 ± 16523.9 |
| Compound (1)-1 mg/kg, p.o. | 188.3 ± 15.6 | 438.5 ± 52.7 | 1797.2 ± 331.3 | 889.5 ± 353.9 | 54241.5 ± 11182.3 |
| Compound (1)-10 mg/kg, p.o. | 93.2 ± 7.9 | 328.9 ± 111.9 | 1463.8 ± 265.3 | 340.4 ± 151.7 | 39050.3 ± 6922.7 |
| Fenofibrate 65 mg/kg, p.o. | 324.9 ± 45.3 | 1048.1 ± 147.4 | 2871.5 ± 357.4 | 1630.0 ± 329.8 | 93412.8 ± 12023.5 |

TABLE NO. 3

| | | | | | % Change in |
|---|---|---|---|---|---|
| | % Change in TG Vs Vehicle Control | | | | AUC-TG vs |
| Treatment Groups | 0 hr | 6 hr | 24 hr | 48 hr | Vehicle Control |
| Vehicle Control | | | | | |
| Compound (1)-1 mg/kg, p.o. | −63.7 ± 3.0 | −43.6 ± 6.8 | −44.7 ± 10.2 | −59.1 ± 16.3 | −48.4 ± 10.6 |
| Compound (1)-10 mg/kg, p.o. | −82.0 ± 1.5 | −57.7 ± 14.4 | −55 0 ± 8.2 | −84.3 ± 7.0 | −62.9 ± 6.6 |
| Fenofibrate 65 mg/kg, p.o. | −37.4 ± 8.7 | 34.9 ± 19.0 | −11.7 ± 11.0 | −25.0 ± 15.2 | −11.2 ± 11.4 |

Compound (1) at 1 and 10 mg/kg showed significant 64% and 82% reduction in serum triglycerides (TG) at 0-hr time points revealed its triglyceride lowering potential after 15 days repeat dose treatment. Triton WR-1339 administration produced significantly increased TG levels at 6 hours onwards, which peaked at 24 hour time point upto 3250 mg/dl. Compound (1) showed dose dependent lowering of TG levels at all-time points. Compound (1) at 1 and 10 mg/kg showed 48 and 63 % reduction in AUC-TG respectively, whereas fenofibrate at 65 mg/kg showed only 11% reduction in AUC-TG Vs Vehicle control group. Therefore Compound (I) was shown to be significantly effective in blunting an increase in serum triglycerides in a model of severe hypertriglyceridemia induced by Triton WR-1339 (by inhibition of LPL) in Zucker fa/fa rats when compared to fenofibrate, a pure PPAR alfa.

### Experiment-2

### Effect of Compound (1) on Plasma Triglycerides Hyper-Chylomicronemia and Chylomicron Dynamics in Zucker fa/fa Rats after 14 Days Repeated Dose Administration

In this study, the effect of Compound (1) on hyper-chylomicronemia and chylomicron dynamics in Zucker fa/fa rats after 14 days repeated dose administration was evaluated.

Zucker (fa/fa) fatty rat of 7-8 weeks of age were purchased from Charles River Laboratory and single-housed under a pathogen free environment with constant room temperature (20-22° C.) and relative humidity (40-50%) and a 6:00 pm-6:00 am dark-light cycle. Rats were provided with free access to food (Lab Diet 5L0D, an irradiated regular rodent diet containing 23% of protein and 4.5% of fat) and water. Initial body weight and body composition (using a NMR analyzer) were measured 6 days post-arrival. The rats were then randomly divided into four dosing groups based BW and fat mass as below—

TABLE NO. 4

| | | | Number of |
|---|---|---|---|
| Sr. no | Treatment Group | Dosage Level | Animals |
| 1 | Vehicle Control [1% | 0 mg/kg | 9 |
| | Tween 20], p.o. | | |
| 2 | Compound (1)-0.4 mg/kg, p.o. | 0.4 mg/kg | 9 |
| 3 | Compound (1)-4 mg/kg, p.o. | 4 mg/kg | 9 |
| 4 | Fenofibrate 150 mg/kg, p.o. | 150 mg/kg | 9 |

Dosing was begun at 9-10 weeks of age, once daily (8-10 am) by oral gavage for 14 days. On the day-14 of dosing, a final body composition (after dosing) was measured. On day-15, all animals were given a fatty tolerance test (FTT) after being fasted overnight (15-16 hours), followed by blood and tissue collection.

Fatty Tolerance Test Procedure (FTT):
1. Following an overnight fast, rats were orally gavaged with a mixture of ¹³C-Palmitic Acid (1 and corn oil (1 g of¹³C-P dissolved in 5 ml of corn oil) at 5 ml/kg·bw.
2. Blood samples were collected via tail vein bleeding using 3×300 ul EDTA tubes at the following time points:

0 hour (initial), 1 hour, 2 hour, 3 hour, 4 hour, 5 hour, 6 hour, 8 hour

After the 8 hour bleeding, rats were knocked down in a drop jar using isoflurane. Once sedated, the rats were hooked up to a Midmark Matrx Anesthesia isoflurane machine that dispensed isoflurane vapor at 3.5-4.5 mL/min with an oxygen flow rate of 1 L/min. Organs and tissues were dissected and frozen immediately in liquid nitrogen and stored at −80° C. LCMS and GCMS were used to assess the incorporation of ¹³C lipids into plasma and tissue lipids (n=5 for tissue metabolomics studies).

Results

The serum triglycerides levels in different treatment groups after fatty tolerance test is given in table no. 5 and ¹³C palmitate uptake in adipose tissue is given in table no. 6

TABLE NO. 5

Plasma TG (mg/dl) in FTT

| Treatment Groups | 0 min | 120 min |
|---|---|---|
| Vehicle Control | 394.4 ± 11.9 | 411.2 ± 41.1 |
| Compound (1)-0.4 mg/kg, p.o. | 330.5 ± 39.2 | 389.2 ± 21.8 |
| Compound (1)-4 mg/kg, p.o. | 84.7 ± 9.3 | 171.3 ± 28.2 |
| Fenofibrate 150 mg/kg, p.o. | 444.7 ± 16.9 | 502.2 ± 8.1 |

| | | | % Change in ¹³C |
|---|---|---|---|
| | Palmitate total | ¹³C Palmitate | Palmitate uptake |
| | (micromole/mg | (Nanomole/mg | in adipose tissue |
| Treatment Groups | protein) | protein) | Vs Vehicle Control |
| Vehicle Control | 6.63 ± 0.13 | 3.34 ± 0.31 | |
| Compound (1)-0.4 mg/kg, p.o. | 7.14 ± 0.30 | 6.20 ± 0.89 | 86 |
| Compound (1)-4 mg/kg, p.o. | 6.60 ± 0.29 | 11.57 ± 0.73 | 247 |
| Fenofibrate 150 mg/kg, p.o. | 6.90 ± 0.24 | 2.88 ± 0.57 | −14 |

From the above study it can be concluded that:
1. Compound (1) significantly reduced plasma triglyceride (TG) at 0 and 2 hour post com oil treatment.
2. The M+16 isotopomers of the major TG species, TG (52:3) and TG (52:4), rose in the first two hour following gavage (reflecting chylomicron production) and declined over the next 4 hours with a secondary rise at 6-8 hour. In contrast, fenofibrate treatment caused a greater increase in M+16 TG species; both low (0.4 mg/kg) and high (4 mg/kg) dose of Compound (1) significantly attenuated the appearance of M+16 TG.
3. In all animals, Major M+16 phosphatidyl choline PC (34:1), carried primarily in HDL and VLDL, rose at similar rates, however the % labeling in the fenofibrate treated animals was significantly lower, suggesting a reduction, in liver derived lipids by fenofibrate.
4. Low and high dose of compound (1) significantly increased the accumulation of M+16 palmitate in adipose tissue (by 86% and 247%, respectively).
5. Both Fenofibrate and compound (1) (low and high dose) decreased gastrocnemius M+16 palmitate labeling, which could be due to induction of lipid oxidation by fenofibrate and potentially compound (1) induced reduced plasma levels of TG in compound (1) treated groups.

In conclusion, compound (1) significantly reduces fasting and postprandial TG levels through enhanced clearance of TG into adipose tissue and works by a mechanism distinct from that of fenofibrate, a ‘pure’ PPARα activator.

1-8. (canceled)

9. A method to delay progression or treatment of a disease or condition, which is selected from chylomicronemia syndrome, familial chylomicronemia syndrome, and Type V hyperlipoproteinemia, comprising administration of a therapeutically effective amount of compound of formula (1) 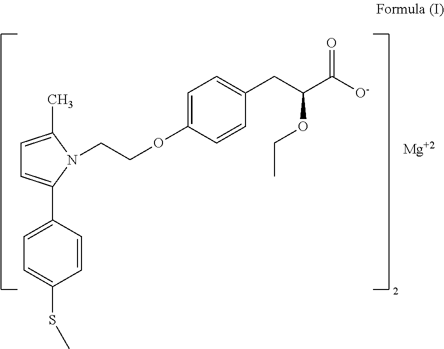
or its pharmaceutical composition to a subject in need of such treatment.

10. A method for the reduction of postprandial triglyceride levels in patients suffering from a disease or condition which is selected from chylomicronemia syndrome, familial chylomicronemia syndrome, and Type V hyperlipoproteinemia, comprising administration of a therapeutically effective amount of compound of formula (1) as claimed in claim 9 to a subject in need of such treatment.

11. A method to delay progression or treatment of pancreatitis in patients suffering from a disease or condition which is selected from chylomicronemia syndrome, familial chylomicronemia syndrome, and type V hyperlipoproteinemia, comprising administration of a therapeutically effective amount of compound of formula (1) as claimed in claim 9 to a subject in need of such treatment.

12. A method to delay progression or treatment of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, comprising administration of a therapeutically effective amount of compound of formula (1) as claimed in claim 9 to a subject in need of such treatment.

13. A method for preventing a disease or condition, which is selected from chylomicronemia syndrome, familial chylomicronemia syndrome, and Type V hyperlipoproteinemia, comprising administration of therapeutically effective amount of compound of formula (1) 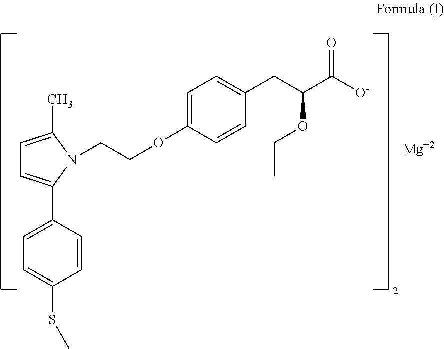
as claimed in claim 9 to a subject in need of such treatment.

14. A method for preventing pancreatitis in a patient suffering from a disease or condition which is selected from chylomicronemia syndrome, familial chylomicronemia syndrome, and Type V hyperlipoproteinemia, comprising administration of a therapeutically effective amount of compound of formula (1) as claimed in claim 9 to a subject in need of such treatment.

15. A method for preventing of a symptom selected from recurrent episodes of pancreatitis, deposition of triglycerides in the skin in the form of eruptive xanthomas, hepatosplenomegaly, milky white triglyceride in the blood vessels in the back of the eye (lipemia retinalis), and mild neuro-cognitive deficits, comprising administration of a therapeutically effective amount of compound of formula (1) as claimed in claim 9 to a subject in need of such treatment.
